(19) United States
(12) Patent Application Publication
(10) Pub. No.: US 2023/0337985 A1
(43) Pub. Date: Oct. 26, 2023

(54) Strain-Sensing Systems, Indwelling Medical Devices, and Methods for Determining Physical Attributes

(71) Applicant: Bard Access Systems, Inc., Salt Lake City, UT (US)

(72) Inventors: Steffan Sowards, Salt Lake City, UT (US); William Robert McLaughlin, Bountiful, UT (US); Anthony K. Misener, Bountiful, UT (US)

(21) Appl. No.: 17/728,802

(22) Filed: Apr. 25, 2022

(51) Int. Cl. A61B 5/00; A61B 5/0205; A61B 5/024; A61B 5/0215; A61B 5/029; A61B 5/08; A61B 5/091

(52) U.S. Cl. CPC A61B 5/6852; A61B 5/0205; A61B 5/02444; A61B 5/02154; A61B 5/029; A61B 5/0816; A61B 5/091; A61B 2562/0266

(57) ABSTRACT

Strain-sensing systems, indwelling medical devices, and methods for determining one or more physical attributes are disclosed. A strain-sensing system can include, for example, an indwelling medical device, an optical interrogator, and a console. The medical device can include an optical-fiber probe having fiber Bragg grating (“FBG”) sensors. The optical interrogator can be configured to send input optical signals into the optical-fiber probe and receive FBG sensor-reflected optical signals therefrom. The console can include one or more processors, memory, and executable instructions that cause the console to perform a set of operations including: receiving the FBG sensor-reflected optical signals from the optical interrogator; converting the FBG sensor-reflected optical signals into converted electrical signals with optical signal-converter logic; and determining in a real-time determination the one-or-more physical attributes associated with a heart, lungs, or both the heart and lungs from at least the converted electrical signals with physical attribute-determination logic.

### BACKGROUND

There is a need for clinicians to be able to easily and safely monitor patients' physical attributes such as those associated with the patients' hearts or lungs. Disclosed herein are strain-sensing systems, indwelling medical devices, and methods for determining such physical attributes.

### SUMMARY

Disclosed herein is a strain-sensing system for determining one or more physical attributes including, in some embodiments, an indwelling medical device, an optical interrogator, a console, and a display screen. The medical device includes an integrated or removable optical-fiber probe having a number of fiber Bragg grating (“FBG”) sensors along at least a distal portion of the optical-fiber probe. The optical interrogator is configured to send input optical signals into the optical-fiber probe as well as receive FBG sensor-reflected optical signals from the optical-fiber probe. The console includes one or more processors, memory, and executable instructions stored in the memory. The executable instructions cause the console to perform a set of operations upon execution of the instructions by the one-or-more processors. The set of operations include receiving the FBG sensor-reflected optical signals from the optical interrogator; converting the FBG sensor-reflected optical signals into converted electrical signals with optical signal-converter logic; and determining in a real-time determination the one-or-more physical attributes associated with a heart, lungs, or both the heart and lungs from at least the converted electrical signals with physical attribute-determination logic. The display screen is configured to display the one-or-more physical attributes associated with the heart, the lungs, or both the heart and lungs.

In some embodiments, the one-or-more physical attributes associated with the heart are selected from heart rate, relative central blood pressure, right atrial pressure, right ventricular pressure, pulmonary artery pressure, pulmonary artery wedge pressure, and stroke volume.

In some embodiments, the one-or-more physical attributes associated with the lungs are selected from respiration rate, vital capacity, tidal volume, inspiratory capacity, expiratory reserve volume, inspiratory reserve volume, and total lung capacity.

In some embodiments, the set of operations further include extracting one or more extracted electrical signals from the converted electrical signals with signal-processing logic. The converted electrical signals include complex oscillations indicative of those experienced by the distal portion of the optical-fiber probe when the optical-fiber probe is disposed in a circulatory system of a patient. The one-or-more extracted electrical signals include one or more simple oscillations indicative of the one-or-more physical attributes associated with the heart, the lungs, or both the heart and lungs.

In some embodiments, the one-or-more physical attributes associated with the heart include heart rate. The physical attribute-determination logic is configured to determine the heart rate directly from a simple oscillation from an extracted electrical signal of the one-or-more extracted electrical signals.

In some embodiments, the one-or-more physical attributes associated with the lungs include respiration rate. The physical attribute-determination logic is configured to determine the respiration rate directly from a simple oscillation from an extracted electrical signal of the one-or-more extracted electrical signals.

In some embodiments, the set of operations further include correlating one or more measurements of the one-or-more physical attributes associated with the heart, the lungs, or both the heart and lungs by one or more measuring devices therefor with the one-or-more simple oscillations indicative of the one-or-more physical attributes associated with the heart, the lungs, or both the heart and lungs. The correlating of the one-or-more measurements of the one-or-more physical attributes with the one-or-more simple oscillations indicative of the one-or-more physical attributes is prior to determining the one-or-more physical attributes associated with the heart, the lungs, or both the heart and lungs in the real-time determination.

In some embodiments, the one-or-more physical attributes associated with the heart are selected from relative central blood pressure, right atrial pressure, right ventricular pressure, pulmonary artery pressure, and pulmonary artery wedge pressure.

In some embodiments, the one-or-more physical attributes associated with the heart include stroke volume.

In some embodiments, the one-or-more physical attributes associated with the lungs are selected from vital capacity, tidal volume, inspiratory capacity, expiratory reserve volume, inspiratory reserve volume, and total lung capacity.

In some embodiments, the set of operations further include establishing one or more physical attribute-baseline measurements through the one-or-more measurements of the one-or-more physical attributes; and monitoring one or more physical-attribute baselines through the one-or-more simple oscillations to determine any deviations from the one-or-more physical-attribute baselines.

In some embodiments, the display screen is configured to display the one-or-more physical attributes associated with the heart, the lungs, or both the heart and lungs in one or more physical-attribute plots to facilitate historical analysis by an attending clinician.

Also disclosed herein is a method of a strain-sensing system for determining one or more physical attributes. The method includes, in some embodiments, a sending step, a first receiving step, a second receiving step, a converting step, and a determining step. The sending step includes sending input optical signals from an optical interrogator into an optical-fiber probe integral with or removably disposed in an indwelling medical device, the optical-fiber probe having a number of fiber Bragg grating (“FBG”) sensors along at least a distal portion of the optical-fiber probe. The first receiving step includes receiving by the optical interrogator FBG sensor-reflected optical signals from the optical-fiber probe. The second receiving step includes receiving by a console the FBG sensor-reflected optical signals from the optical interrogator. The console includes one or more processors, memory, and executable instructions stored in the memory that cause the console to perform various operations of the method upon execution of the instructions by the one-or-more processors. The converting step includes converting the FBG sensor-reflected optical signals into converted electrical signals with optical signal-converter logic. The determining step includes determining in a real-time determination the one-or-more physical attributes associated with a heart, lungs, or both the heart and lungs from at least the converted electrical signals with physical attribute-determination logic.

In some embodiments, the method further includes an extracting step. The extracting step includes extracting one or more extracted electrical signals from the converted electrical signals with signal-processing logic. The converted electrical signals include complex oscillations indicative of those experienced by the distal portion of the optical-fiber probe when the optical-fiber probe is disposed in a circulatory system of a patient. The one-or-more extracted electrical signals include one or more simple oscillations indicative of the one-or-more physical attributes associated with the heart, the lungs, or both the heart and lungs.

In some embodiments, the one-or-more physical attributes associated with the heart include heart rate. The physical attribute-determination logic is configured to determine the heart rate directly from a simple oscillation from an extracted electrical signal of the one-or-more extracted electrical signals.

In some embodiments, the one-or-more physical attributes associated with the lungs include respiration rate. The physical attribute-determination logic is configured to determine the respiration rate directly from a simple oscillation from an extracted electrical signal of the one-or-more extracted electrical signals.

In some embodiments, the method further includes a correlating step. The correlating step includes correlating one or more measurements of the one-or-more physical attributes associated with the heart, the lungs, or both the heart and lungs by one or more measuring devices therefor with the one-or-more simple oscillations indicative of the one-or-more physical attributes associated with the heart, the lungs, or both the heart and lungs. The correlating step of correlating of the one-or-more measurements of the one-or-more physical attributes with the one-or-more simple oscillations indicative of the one-or-more physical attributes is prior to the determining step of determining the one-or-more physical attributes associated with the heart, the lungs, or both the heart and lungs in the real-time determination.

In some embodiments, the one-or-more physical attributes associated with the heart are selected from relative central blood pressure, right atrial pressure, right ventricular pressure, pulmonary artery pressure, and pulmonary artery wedge pressure.

In some embodiments, the one-or-more physical attributes associated with the heart include stroke volume.

In some embodiments, the one-or-more physical attributes associated with the lungs are selected from vital capacity, tidal volume, inspiratory capacity, expiratory reserve volume, inspiratory reserve volume, and total lung capacity.

In some embodiments, the method further includes an establishing step and a monitoring step. The establishing step includes establishing one or more physical attribute-baseline measurements through the one-or-more measurements of the one-or-more physical attributes. The monitoring step includes monitoring one or more physical-attribute baselines through the one-or-more simple oscillations to determine any deviations from the one-or-more physical-attribute baselines.

In some embodiments, the method further includes a displaying step. The displaying step includes displaying the physical attributes associated with the heart, the lungs, or both the heart and lungs on a display screen, optionally, in one or more physical-attribute plots to facilitate historical analysis by an attending clinician.

These and other features of the concepts provided herein will become more apparent to those of skill in the art in view of the accompanying drawings and following description, which describe particular embodiments of such concepts in greater detail.

### DRAWINGS

FIG. 1 illustrates a strain-sensing system in use on a patient in accordance with some embodiments.

FIG. 2 illustrates a detailed view of the strain-sensing system in accordance with some embodiments.

FIG. 3 illustrates a block diagram of the strain-sensing system including a console, a stand-alone optical interrogator, and a stand-alone display in accordance with some embodiments.

FIG. 4 illustrates a block diagram of the strain-sensing system including a console with an integrated optical interrogator and an integrated display screen in accordance with some embodiments.

FIG. 5 illustrates a transverse cross-section of a catheter tube of a catheter including an integrated optical-fiber probe in accordance with some embodiments.

FIG. 6 illustrates a longitudinal cross-section of the catheter tube in accordance with some embodiments.

FIG. 7 provides a number of different plots on a display screen of the strain-sensing system in accordance with some embodiments.

FIG. 8 provides a detailed plot of curvature vs. arc length and torsion vs. arc length for at least a distal portion of the optical-fiber probe as one of the plots of FIG. 7.

FIG. 9 provides a detailed plot of angle vs. arc length for at least the distal portion of the optical-fiber probe as one of the plots of FIG. 7.

FIG. 10 provides a detailed plot of position vs. time for at least the distal portion of the optical-fiber probe as one of the plots of FIG. 7.

FIG. 11 provides a displayable shape for at least the distal portion of the optical-fiber probe in accordance with some embodiments.

FIG. 12 provides detailed plots of curvature vs. time for each FBG sensor selected from a number of FBG sensors of the optical-fiber probe as some of the plots of FIG. 7.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

The term “logic” can be representative of hardware, firmware, or software that is configured to perform one or more functions. As hardware, logic can include circuitry having data processing or data storage functionality. An example of such circuitry can include, but is not limited to, a hardware processor (e.g., a microprocessor, one or more processor cores, a digital-signal processor [“DSP”], a programmable gate array [“PGA”], a microcontroller, an application-specific integrated circuit [“ASIC”], etc.) or semiconductor memory. As firmware, the logic can be stored in persistent storage. As software, logic can include one or more processes, instances, Application Programming Interfaces (“APIs”), subroutines, functions, applets, servlets, or routines. Logic can also include source code, object code, a shared library, a dynamic link library (“DLL”), or even one or more instructions. Such software can be stored in any type of suitable non-transitory storage medium or transitory storage medium (e.g., electrical, optical, acoustical, or any other form of propagated signal including carrier waves, infrared signals, or digital signals). An example of a non-transitory storage medium can include, but is not limited to, a programmable circuit; non-persistent storage such as volatile memory (e.g., any type of random-access memory [“RAM”]); or persistent storage such as non-volatile memory (e.g., read-only memory [“ROM”], power-backed RAM, flash memory, phase-change memory, etc.), a solid-state drive, a hard-disk drive, an optical-disc drive, or a portable memory device.

Unless defined otherwise, all technical and scientific terms used herein have the same meaning as commonly understood by those of ordinary skill in the art.

As set forth above, there is a need for clinicians to be able to easily and safely monitor patients' physical attributes such as those associated with the patients' hearts or lungs. Disclosed herein are strain-sensing systems, indwelling medical devices, and methods for determining such physical attributes.

For example, a strain-sensing system for determining one or more physical attributes can include an indwelling medical device, an optical interrogator, a console, and a display. The medical device can include an optical-fiber probe having a number of fiber Bragg grating (“FBG”) sensors along at least a distal portion of the optical-fiber probe. The optical interrogator can be configured to send input optical signals into the optical-fiber probe as well as receive FBG sensor-reflected optical signals from the optical-fiber probe. The console can include one or more processors, memory, and executable instructions stored in the memory. The executable instructions can cause the console to perform a set of operations upon execution of the instructions by the one-or-more processors. The set of operations can include receiving the FBG sensor-reflected optical signals from the optical interrogator; converting the FBG sensor-reflected optical signals into converted electrical signals with optical signal-converter logic; and determining in a real-time determination the one-or-more physical attributes associated with a heart, lungs, or both the heart and lungs from at least the converted electrical signals with physical attribute-determination logic. The display screen can be configured to display the one-or-more physical attributes associated with the heart, the lungs, or both the heart and lungs.

These and other features of the strain-sensing systems and methods provided herein will become more apparent with reference to the accompanying drawings and the following description, which provide particular embodiments of the strain-sensing systems, indwelling medical devices, and methods for determining one or more physical attributes in greater detail.

### Strain-Sensing Systems

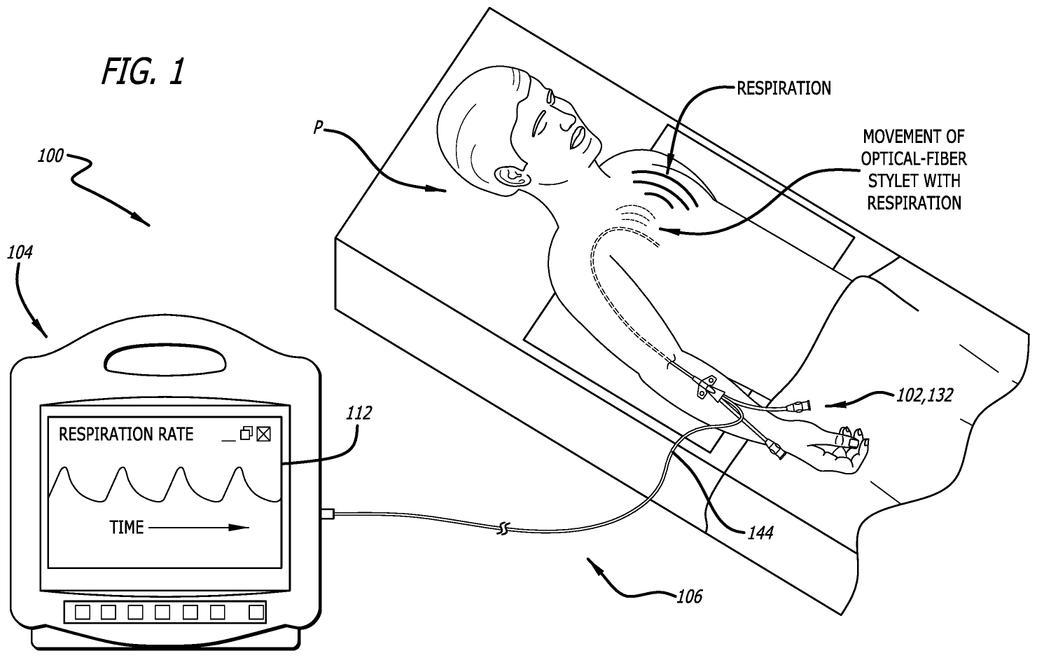

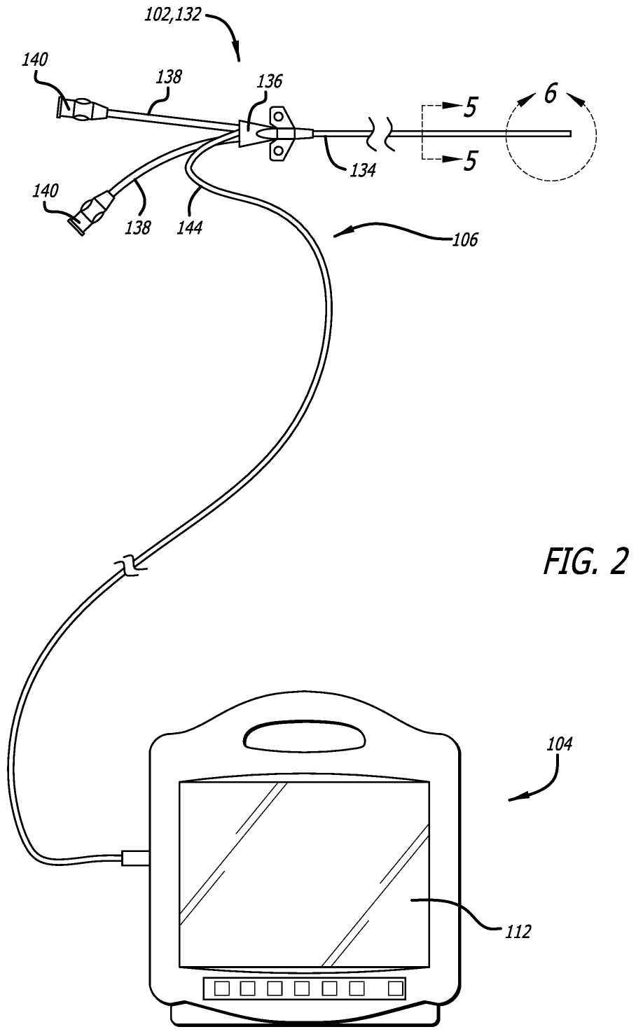

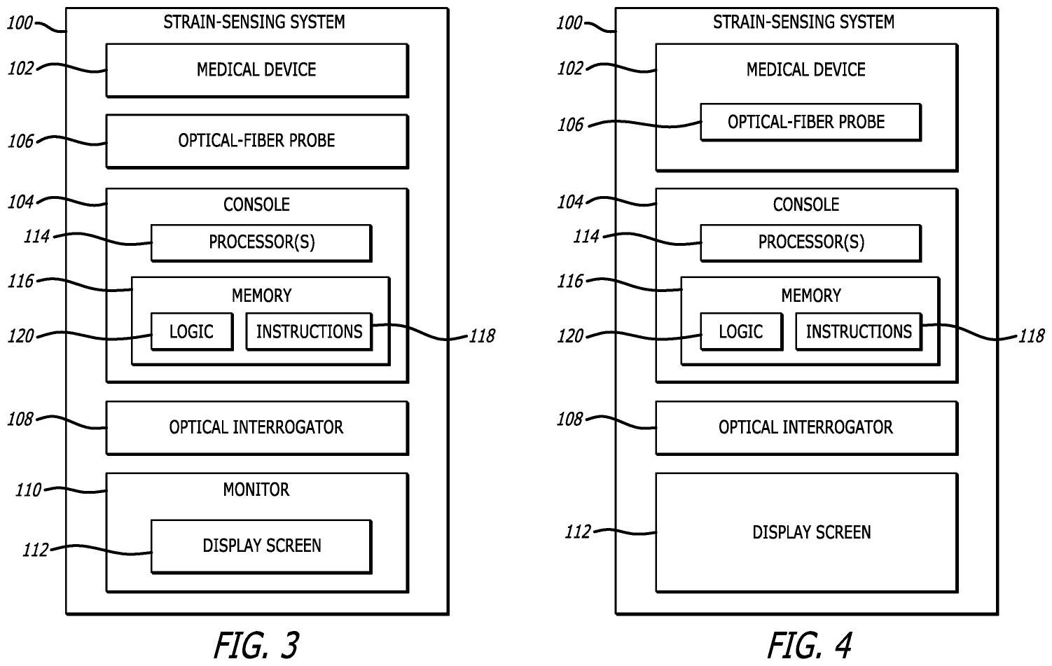

FIG. 1 illustrates a strain-sensing system 100 in use on a patient P in accordance with some embodiments. FIG. 2 illustrates a detailed view of the strain-sensing system 100 in accordance with some embodiments. FIGS. 3 and 4 illustrate block diagrams of the strain-sensing system 100 in accordance with different embodiments thereof.

The strain-sensing system 100 is configured for determining one or more physical attributes of the patient P such as any one or more physical attributes associated with a heart, lungs, or both the heart and lungs of the patient P. Thus, the strain-sensing system 100 can include at least an indwelling medical device 102, an optical-fiber probe 106 removably disposed in the medical device 102, a console 104, an optical interrogator 108 configured to stand alone, and a monitor 110 including a display screen 112 as shown in FIG. 3. Alternatively, as shown in FIG. 4, the strain-sensing system 100 can include at least the medical device 102 and the console 104, wherein the medical device 102 includes the optical-fiber probe 106 integrated in the medical device 102 and the console 104 includes both the optical interrogator 108 and the display screen 112 integrated in the console 104. Notably, the strain-sensing system 100 of FIG. 3 is mostly separated in terms of components of the strain-sensing system 100. In contrast, the strain-sensing system 100 of FIG. 4 is mostly integrated in terms of components of the strain-sensing system 100. However, it should be understood additional strain-sensing systems are possible between at least the foregoing separated and integrated embodiments of the strain-sensing system 100. For example, the strain-sensing system 100 can include the medical device 102, the removable optical-fiber probe 106, and the console 104 with the integrated display screen 112 but without the integrated optical interrogator 108. Indeed, such a strain-sensing system can include the stand-alone optical interrogator 108. While not shown, the strain-sensing system 100 can further include an optical-fiber connector module configured for connecting the optical-fiber probe 106, a single-use disposable component of the strain-sensing system 100, to a remainder of the strain-sensing system 100 such as the stand-alone optical interrogator 108 or the console 104 including the integrated optical interrogator 108, which are multi-use capital components of the strain-sensing system 100.

The medical device 102 is set forth below in its own section in which particular embodiments of the medical device 102 are provided such as that of the peripherally inserted central catheter (“PICC”) 132.

The console 104 includes one or more processors 114, memory 116, and executable instructions 118 stored in the memory 116 that, upon execution of the instructions 118 by the one-or-more processors 114, cause the console 104 to perform a set of operations in support of determining the one-or-more physical attributes of the patient P. Indeed, the set of operations can include receiving FBG sensor-reflected optical signals from the optical interrogator 108; converting the FBG sensor-reflected optical signals into converted electrical signals with the optical signal-converter logic, wherein the converted electrical signals include complex oscillations indicative of those experienced by the distal portion of the optical-fiber probe 106 when the optical-fiber probe 106 is disposed in a circulatory system of the patient P; extracting one or more extracted electrical signals from the converted electrical signals with the signal-processing logic, wherein the one-or-more extracted electrical signals include one or more simple oscillations or one or more compound oscillations (e.g., two or more simple oscillations added together) thereof indicative of the one-or-more physical attributes of the patient P; correlating one or more measurements of the one-or-more physical attributes of the patient P by one or more measuring devices therefor with the one-or-more simple or compound oscillations indicative of the one-or-more physical attributes of the patient P; and determining in a real-time determination the one-or-more physical attributes of the patient P from the foregoing signals with the physical attribute-determination logic.

As alluded to above, the console 104 also includes logic 120 such as optical signal-converter logic, signal-processing logic, and physical attribute-determination logic. The optical signal-converter logic is configured to convert the FBG sensor-reflected optical signals from the optical-fiber probe 106 into plottable data for displayable shapes on the display screen 112 corresponding to the medical device 102 in which the optical-fiber probe 106 is integrated or removably disposed. The optical signal-convertor logic is also configured to convert the FBG sensor-reflected optical signals from the optical-fiber probe 106 into plottable data for a number of other plots of the plottable data on the display screen 112. The optical signal-converter logic is also configured to convert the FBG sensor-reflected optical signals received from the optical interrogator 108 into the converted electrical signals. The signal-processing logic is configured to extract the one-or-more extracted electrical signals from the converted electrical signals. Notably, the converted electrical signals include the complex oscillations indicative of those experienced by the distal portion of the optical-fiber probe 106 when the optical-fiber probe 106 is disposed in the circulatory system of the patient P, and the one-or-more extracted electrical signals include the one-or-more simple or compound oscillations indicative of the one-or-more physical attributes of the patient P. The physical attribute-determination logic is configured to determine in a real-time determination the one-or-more physical attributes of the patient P from the one-or-more extracted electrical signals. In an example, the physical attribute-determination logic is configured to directly determine the heart rate from a simple or compound oscillation from an extracted electrical signal of the one-or-more extracted electrical signals, wherein the simple or compound oscillation has a period corresponding to that of the heart beating. In another example, the physical attribute-determination logic is configured to directly determine the respiration rate directly from a simple or compound oscillation from an extracted electrical signal of the one-or-more extracted electrical signals, wherein the simple or compound oscillation has a period corresponding to that of the lungs breathing.

Notably, the one-or-more physical attributes of the patient P that the strain-sensing system 100 is configured to determine include those associated with the heart, the lungs, or both the heart and lungs of the patient P. In an example, the one-or-more physical attributes associated with the heart are selected from heart rate, relative central blood pressure, right atrial pressure, right ventricular pressure, pulmonary artery pressure, pulmonary artery wedge pressure, and stroke volume, which is the volume of blood pumped from the left ventricle of the heart per beat as determined by the relative difference in ending diastolic volume and ending systolic volume. In another example, the one-or-more physical attributes associated with the lungs are selected from respiration rate, vital capacity, tidal volume, inspiratory capacity, expiratory reserve volume, inspiratory reserve volume, and total lung capacity. While the physical attribute-determination logic is configured to directly determine the heart rate or the respiration rate of the patient P from a simple or compound oscillation from an extracted electrical signal indicative of the heart of the patient P beating or the lungs of the patient P respiring, the physical attribute-determination logic is also configured to determine the foregoing pressures and volumes of the heart as well as the capacities and volumes of the lungs from simple or compound oscillations correlated with the one-or-more measurements of the one-or-more physical attributes of the patient P by the one-or-more measuring devices. Indeed, the correlating of the one-or-more measurements of the one-or-more physical attributes with the one-or-more simple or compound oscillations indicative of the one-or-more physical attributes is performed prior to the determining of the one-or-more physical attributes of the patient P in the real-time determination. Furthermore, the set of operations set forth above can further include establishing one or more physical attribute-baseline measurements through the one-or-more measurements of the one-or-more physical attributes, as well as monitoring one or more physical-attribute baselines through the one-or-more simple oscillations to determine any deviations from the one-or-more physical-attribute baselines.

The optical interrogator 108 is configured to send input optical signals (e.g., 1460-1620 nm laser light by way of a tunable laser) into the optical-fiber probe 106 and receive the FBG sensor-reflected optical signals from the optical-fiber probe 106. When the optical-fiber connector module is present in the strain-sensing system 100, the optical interrogator 108 is configured to send the input optical signals into the optical-fiber probe 106 of the medical device 102 by way of the optical-fiber connector module and receive the FBG sensor-reflected optical signals from the optical-fiber probe 106 by way of the optical-fiber connector module.

The display screen 112 is configured to display the one-or-more physical attributes associated with the heart, the lungs, or both the heart and lungs of the patient P. The display screen 112 is also configured to display the one-or-more physical attributes associated with the heart, the lungs, or both the heart and lungs in one or more physical-attribute plots to facilitate historical analysis by an attending clinician. (See, for example, the plot of respiration rate on the display screen 112 of the console 104 of FIG. 1.) The display screen 112 is also configured to display shapes on the display screen 112 corresponding to the medical device 102 in which the optical-fiber probe 106 is integrated or removably disposed as well as the number of other plots of the plottable data alluded to above on the display screen 112.

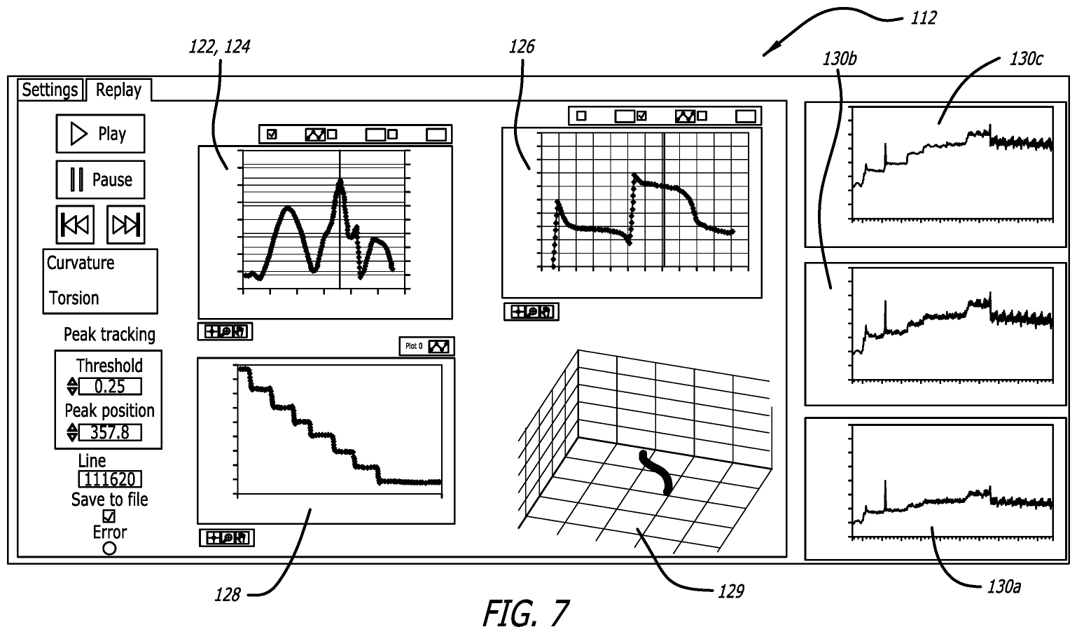

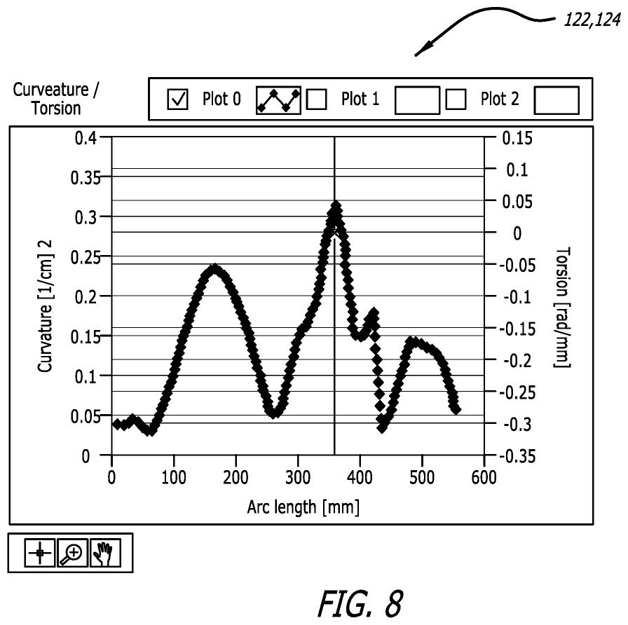

FIG. 7 provides the display screen 112 of the strain-sensing system 100 in accordance with some embodiments. FIGS. 8-12 provide detailed plots of the number of different plots on the display screen 112 of FIG. 7.

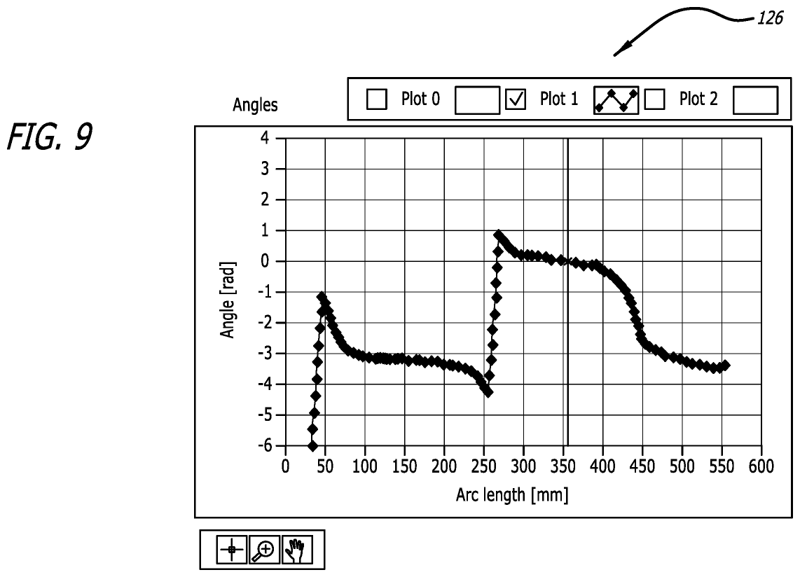

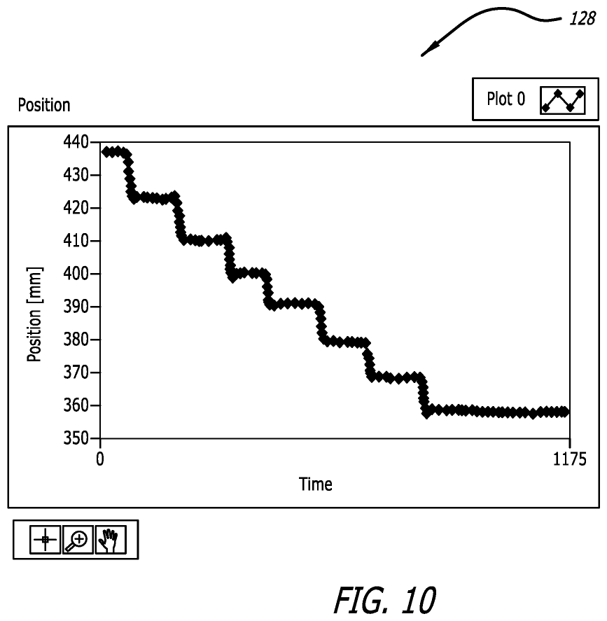

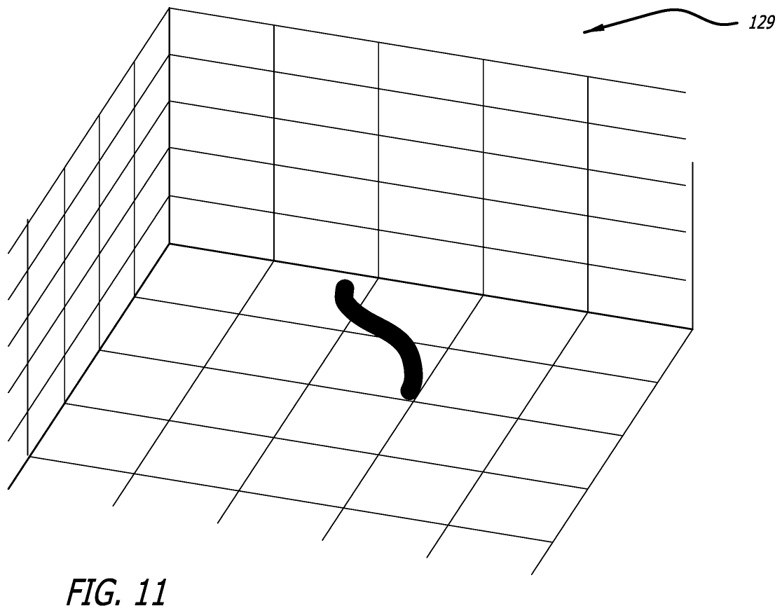

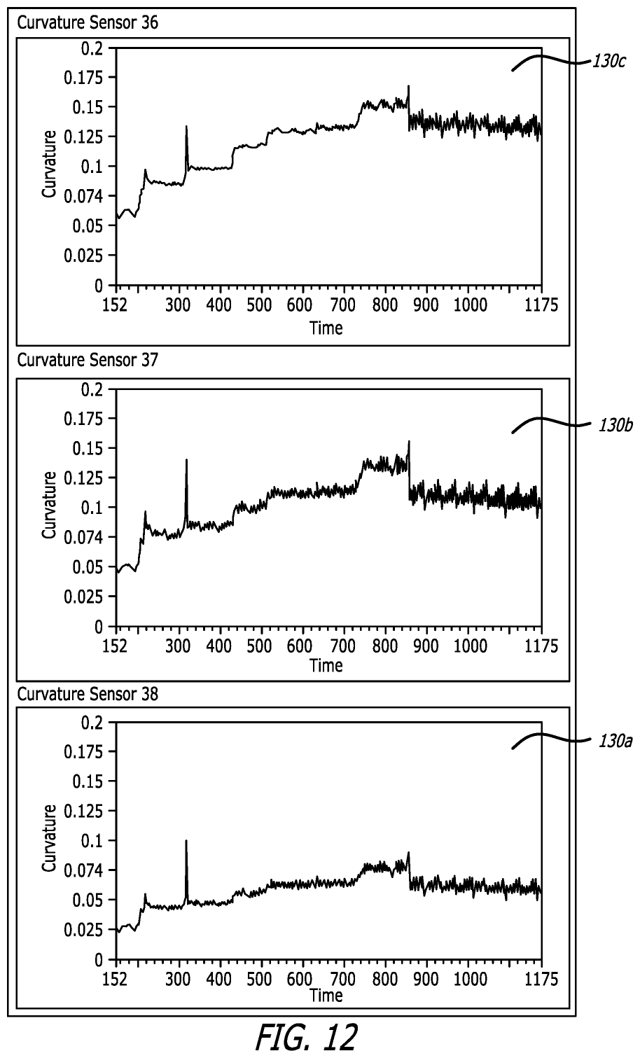

The number of other plots can include a plot of curvature vs. arc length 122 as shown in FIG. 8, a plot of torsion vs. arc length 124 as also shown in FIG. 8, a plot of angle vs. arc length 126 as shown in FIG. 9, or a plot of position vs. time 128 as shown in FIG. 10 for at least the distal portion of the optical-fiber probe 106. The number of other plots can further include a shape for the medical device 102 over a 3-dimensional grid 129. The number of other plots can even further include plots of curvature vs. time 130a, 130b, and 130c as shown in FIG. 12 for a selection of the FBG sensors such as the FBG sensors 146a, 146b, and 146c in the distal portion of the optical-fiber probe 106. Any one or more of the plots of curvature vs. time 130a, 130b, and 130c for the selection of the FBG sensors 146a, 146b, and 146c in the distal portion of the optical-fiber probe 106 can be used to manually identify a distinctive change in curvature of the optical-fiber probe 106 by way of a distinctive change (e.g., increase followed by decrease in curvature at about 860 s) in plotted curvature of the optical-fiber probe 106 at a moment a tip of the medical device 102 is advanced into a superior vena cava (“SVC”) of the patient P. However, the three plots of curvature vs. time 130a, 130b, and 130c shown in FIGS. 7 and 12 are those for a last three FBG sensors, namely the FBG sensors 146a, 146b, and 146c, in the distal portion of the optical-fiber probe 106. The last three FBG sensors 146a, 146b, and 146c in the distal portion of the optical-fiber probe 106 are particularly useful in identifying the distinctive change in the plotted curvature of the optical-fiber probe 106 in that the foregoing FBG sensors 146a, 146b, and 146c directly experience a physical change in curvature resulting from tensile strain and compressive strain of the optical-fiber probe 106 when the tip of the medical device 102 is advanced into the SVC of the patient P. The distinctive change in the plotted curvature of the optical-fiber probe 106 is exemplified by an instantaneous increase in the plotted curvature followed by an instantaneous decrease in the plotted curvature having a magnitude about twice that of the instantaneous increase in the plotted curvature as shown in each plot of curvature vs. time 130a, 130b, and 130c in FIG. 12 at about 860 s.

In addition to being able to use any one or more of the plots of curvature vs. time 130a, 130b, and 130c to manually identify the distinctive change in the curvature of the optical-fiber probe 106 at the moment the tip of the medical device 102 is advanced into the SVC of the patient P, any one or more of the plots of curvature vs. time 130a, 130b, and 130c for the selection of the FBG sensors, namely the FBG sensors 146a, 146b, and 146c, in the distal portion of the optical-fiber probe 106 can be used to manually confirm the tip of the medical device 102 is in the SVC by way of oscillations in the tip of the optical-fiber probe 106. The oscillations in the tip of the optical-fiber probe 106 are evidenced in the plotted curvature of the optical-fiber probe 106 sensed by the selection of the FBG sensors 146a, 146b, and 146c. (See the three plots of curvature vs. time 130a, 130b, and 130c in FIGS. 7 and 12, between about 860 s and 1175 s when the distal portion of the optical-fiber probe 106 is held in position in the SVC as shown by the plot of position vs. time.) The oscillations in the tip of the optical-fiber probe 106 result from changes in blood flow within the SVC sensed by the selection of the FBG sensors 146a, 146b, and 146c as a heart of the patient P beats.

When present, the optical-fiber connector module includes a housing, a receptacle disposed in the housing, a cable extending from the housing, a plug in a free terminus of the cable, and an optical fiber within the cable extending from the receptacle to the plug. The optical-fiber connector module is configured to establish a first optical connection between the optical-fiber probe 106 and the optical fiber of the optical-fiber connector module. Indeed, the receptacle includes an optical receiver configured to accept insertion of an optical terminal of a plug of the optical-fiber probe 106 for establishing an optical connection between the optical-fiber connector module and the optical-fiber probe 106 of the medical device 102 when the plug is inserted into the receptacle. Likewise, the optical-fiber connector module is configured to establish a second optical connection between the optical fiber of the optical-fiber connector module and the optical interrogator 108. The optical fiber of the optical-fiber connector module is configured to convey the input optical signals from the optical interrogator 108 to the optical-fiber probe 106 and the FBG sensor-reflected optical signals from the optical-fiber probe 106 to the optical interrogator 108.

The optical-fiber connector module can further include one or more sensors selected from at least a gyroscope, an accelerometer, and a magnetometer disposed within the housing. The one-or-more sensors are configured to provide sensor data to the console 104 or by way of one or more data wires within at least the cable for determining a reference plane with reference plane-determiner logic of the logic 120 for strain sensing with the optical-fiber probe 106.

The optical-fiber connection module is configured to sit within a fenestration of a surgical drape adjacent a percutaneous insertion site for the medical device 102. As the optical-fiber connection module is configured to sit within the fenestration of the surgical drape, the optical-fiber connection module is amenable to disinfection or sterilization. For example, the housing of the optical-fiber connection module can be non-porous or chemically resistant to oxidants. The optical-fiber connection module can be configured for manual disinfection with a ChloraPrep® product by Becton, Dickinson and Company (Franklin Lakes, NJ), or the optical-fiber connection module can be configured for automatic high-level disinfection or sterilization with vaporized H₂O₂ by way of Trophon® by Nanosonics Inc. (Indianapolis, IN).

### Medical Devices

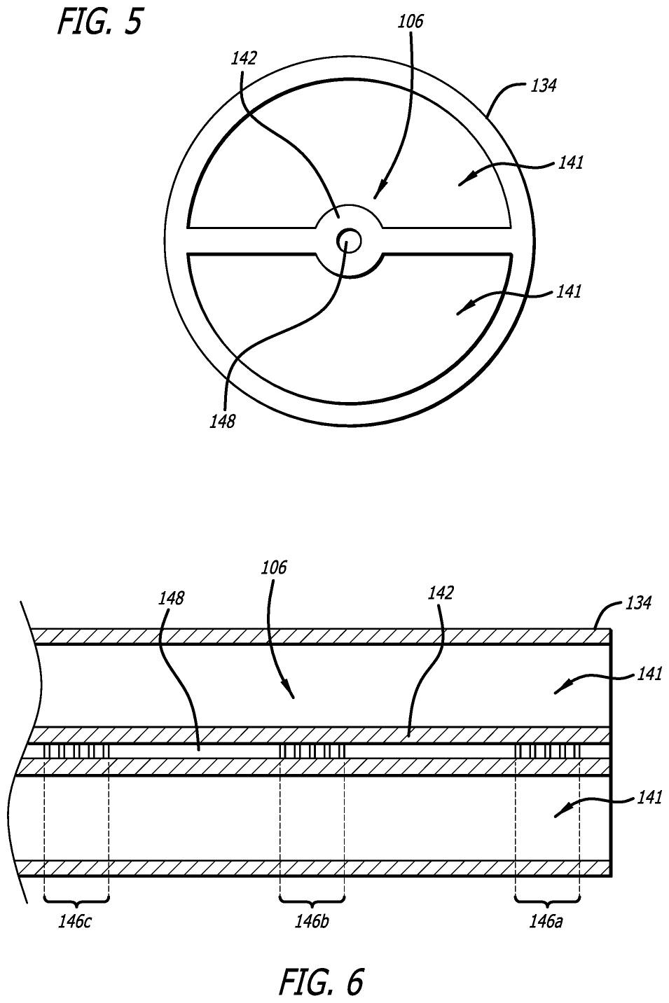

FIG. 2 illustrates a PICC 132 as the medical device 102 of the strain-sensing system 100 in accordance with some embodiments. FIG. 5 illustrates a transverse cross-section of a catheter tube 134 of the PICC 132 including the integrated optical-fiber probe 106 in accordance with some embodiments. FIG. 6 illustrates a longitudinal cross-section of the catheter tube 134 of the PICC 132 including the integrated optical-fiber probe 106 in accordance with some embodiments.

As shown, the PICC 132 includes the catheter tube 134, a bifurcated hub 136, two extension legs 138, and two Luer connectors 140 operably connected in the foregoing order. The catheter tube 134 includes two catheter-tube lumens 141 and the optical-fiber probe 106 disposed in a longitudinal bead 142 of the catheter tube 134 such as between the two catheter-tube lumens 141, as extruded. Optionally, in a same or different longitudinal bead of the catheter tube 134, the PICC 132 can further include an electrocardiogram (“ECG”) stylet, which can provide ECG data complementary to FBG-sensor data for determining the one-or-more physical attributes of the patient P. The bifurcated hub 136 has two hub lumens correspondingly fluidly connected to the two catheter-tube lumens. Each extension leg of the two extension legs 138 has an extension-leg lumen fluidly connected to a hub lumen of the two hub lumens. The PICC 132 further includes a stylet extension tube 144 extending from the bifurcated hub 136. The stylet extension tube 144 can be a skived portion of the catheter tube 134 including the optical-fiber probe 106 or the skived portion of the catheter tube 134 disposed in another tube, either of which can terminate in a plug for establishing an optical connection between the optical fiber of the optical-fiber connector module and the optical-fiber probe 106 of the PICC 132.

Notably, when the optical-fiber probe 106 is removable, the PICC 132 includes at least a trifurcated hub and the catheter tube 134 includes three catheter-tube lumens. The trifurcated hub has three hub lumens correspondingly fluidly connected to the three catheter-tube lumens with the optical-fiber probe 106 removably disposed in a lumen of the foregoing lumens as shown in FIG. 2. However, the foregoing PICC 132 can further include a third extension leg having an extension-leg lumen fluidly connected to a hub lumen of the three hub lumens. When the PICC 132 includes the third extension leg, the optical-fiber can be further disposed therein.

The optical-fiber probe 106, which can include a single core or multiple cores, includes a number of FBG sensors 146a, 146b, 146c, . . . , 146n along at least a distal portion of the optical-fiber probe 106 configured for strain sensing with the strain-sensing system 100. The FBG sensors 146a, 146b, 146c, . . . , 146n include variations in refractive index of an optical fiber 148 of the optical-fiber probe 106, thereby forming wavelength-specific reflectors of the FBG sensors 146a, 146b, 146c, . . . , 146n configured to reflect the input optical signals sent into the optical-fiber probe 106 by the optical interrogator 108. FIG. 6 illustrates, in particular, a last three FBG sensors 146a, 146b, and 146c in the distal portion of the optical-fiber probe 106, which FBG sensors 146a, 146b, and 146c are particularly useful in identifying the distinctive change in the plotted curvature of the optical-fiber probe 106 as set forth above. This is because the last three FBG sensors 146a, 146b, and 146c directly experience a physical change in curvature of the optical-fiber probe 106 when, in this case, a tip of the PICC 132 is advanced into the SVC of the patient P.

While the PICC 132 is provided as a particular embodiment of the medical device 102 of the strain-sensing system 100, it should be understood that any medical device of a number of medical devices including catheters such as a CVC can include the optical-fiber probe 106.

### Methods

Methods include a method of the strain-sensing system 100 for determining one or more physical attributes of the patient P. Such a method includes one or more steps selected from a sending step, a first receiving step, a second receiving step, a converting step, an extracting step, a correlating step, an establishing step, a determining step, a monitoring step, and a displaying step.

The sending step includes sending the input optical signals from the optical interrogator 108 into the optical-fiber probe 106 integral with or removably disposed in the indwelling medical device 102. As set forth above, the optical-fiber probe 106 has the number of FBG sensors 146a, 146b, 146c, . . . , 146n along at least the distal portion of the optical-fiber probe 106.

The first receiving step includes receiving by the optical interrogator 108 FBG sensor-reflected optical signals from the optical-fiber probe 106.

The second receiving step includes receiving by the console 104 the FBG sensor-reflected optical signals from the optical interrogator 108. The console 104 includes the one-or-more processors 114, the memory 116, and the executable instructions 118 stored in the memory 116 that cause the console 104 to perform the set of operations set forth above upon execution of the instructions 118 by the one-or-more processors 114.

The converting step includes converting the FBG sensor-reflected optical signals into the converted electrical signals with the optical signal-converter logic.

The extracting step includes extracting the one-or-more extracted electrical signals from the converted electrical signals with the signal-processing logic. The converted electrical signals include the complex oscillations indicative of those experienced by the distal portion of the optical-fiber probe 106 when the optical-fiber probe 106 is disposed in the circulatory system of the patient P. The one-or-more extracted electrical signals include the one-or-more simple or compound oscillations indicative of the one-or-more physical attributes associated with the heart, the lungs, or both the heart and lungs of the patient P.

The correlating step includes correlating one or more measurements of the one-or-more physical attributes associated with the heart, the lungs, or both the heart and lungs by one or more measuring devices therefor with the one-or-more simple or compound oscillations indicative of the one-or-more physical attributes associated with the heart, the lungs, or both the heart and lungs of the patient P. The correlating step is performed prior to the determining step in the real-time determination.

The establishing step includes establishing one or more physical attribute-baseline measurements through the one-or-more measurements of the one-or-more physical attributes.

The determining step includes determining in a real-time determination the one-or-more physical attributes associated with the heart, the lungs, or both the heart and lungs of the patient P from at least the converted electrical signals with the physical attribute-determination logic.

The monitoring step includes monitoring one or more physical-attribute baselines through the one-or-more simple oscillations to determine any deviations from the one-or-more physical-attribute baselines.

The displaying step includes displaying the physical attributes associated with the heart, the lungs, or both the heart and lungs of the patient P on the display screen 112, optionally, in one or more physical-attribute plots to facilitate historical analysis by an attending clinician.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures can be made from the particular embodiments disclosed herein without departing from the scope of the concepts provided herein.

What is claimed is:

1. A strain-sensing system for determining one or more physical attributes, comprising:
an indwelling medical device including an integrated or removable optical-fiber probe having a number of fiber Bragg grating (“FBG”) sensors along at least a distal portion of the optical-fiber probe;
an optical interrogator configured to send input optical signals into the optical-fiber probe and receive FBG sensor-reflected optical signals from the optical-fiber probe;
a console including one or more processors, memory, and executable instructions stored in the memory that cause the console to perform a set of operations upon execution of the instructions by the one-or-more processors, the set of operations including:
receiving the FBG sensor-reflected optical signals from the optical interrogator;
converting the FBG sensor-reflected optical signals into converted electrical signals with optical signal-converter logic; and
determining in a real-time determination the one-or-more physical attributes associated with a heart, lungs, or both the heart and lungs from at least the converted electrical signals with physical attribute-determination logic; and
a display screen configured to display the one-or-more physical attributes associated with the heart, the lungs, or both the heart and lungs.

2. The strain-sensing system of claim 1, wherein the one-or-more physical attributes associated with the heart are selected from heart rate, relative central blood pressure, right atrial pressure, right ventricular pressure, pulmonary artery pressure, pulmonary artery wedge pressure, and stroke volume.

3. The strain-sensing system of claim 1, wherein the one-or-more physical attributes associated with the lungs are selected from respiration rate, vital capacity, tidal volume, inspiratory capacity, expiratory reserve volume, inspiratory reserve volume, and total lung capacity.

4. The strain-sensing system of claim 1, the set of operations further including extracting one or more extracted electrical signals from the converted electrical signals with signal-processing logic, the converted electrical signals including complex oscillations indicative of those experienced by the distal portion of the optical-fiber probe when disposed in a circulatory system of a patient and the one-or-more extracted electrical signals including one or more simple oscillations indicative of the one-or-more physical attributes associated with the heart, the lungs, or both the heart and lungs.

5. The strain-sensing system of claim 4, wherein the one-or-more physical attributes associated with the heart include heart rate, the physical attribute-determination logic configured to determine the heart rate directly from a simple oscillation from an extracted electrical signal of the one-or-more extracted electrical signals.

6. The strain-sensing system of claim 4, wherein the one-or-more physical attributes associated with the lungs include respiration rate, the physical attribute-determination logic configured to determine the respiration rate directly from a simple oscillation from an extracted electrical signal of the one-or-more extracted electrical signals.

7. The strain-sensing system of claim 6, the set of operations further including correlating one or more measurements of the one-or-more physical attributes associated with the heart, the lungs, or both the heart and lungs by one or more measuring devices therefor with the one-or-more simple oscillations indicative of the one-or-more physical attributes associated with the heart, the lungs, or both the heart and lungs prior to determining the one-or-more physical attributes associated with the heart, the lungs, or both the heart and lungs in the real-time determination.

8. The strain-sensing system of claim 7, wherein the one-or-more physical attributes associated with the heart are selected from relative central blood pressure, right atrial pressure, right ventricular pressure, pulmonary artery pressure, and pulmonary artery wedge pressure.

9. The strain-sensing system of claim 7, wherein the one-or-more physical attributes associated with the heart include stroke volume.

10. The strain-sensing system of claim 7, wherein the one-or-more physical attributes associated with the lungs are selected from vital capacity, tidal volume, inspiratory capacity, expiratory reserve volume, inspiratory reserve volume, and total lung capacity.

11. The strain-sensing system of claim 7, the set of operations further including:
establishing one or more physical attribute-baseline measurements through the one-or-more measurements of the one-or-more physical attributes; and
monitoring one or more physical-attribute baselines through the one-or-more simple oscillations to determine any deviations from the one-or-more physical-attribute baselines.

12. The strain-sensing system of claim 1, wherein the display screen is configured to display the one-or-more physical attributes associated with the heart, the lungs, or both the heart and lungs in one or more physical-attribute plots to facilitate historical analysis by an attending clinician.

13. A method of a strain-sensing system for determining one or more physical attributes, comprising:
sending input optical signals from an optical interrogator into an optical-fiber probe integral with or removably disposed in an indwelling medical device, the optical-fiber probe having a number of fiber Bragg grating (“FBG”) sensors along at least a distal portion of the optical-fiber probe;
receiving by the optical interrogator FBG sensor-reflected optical signals from the optical-fiber probe;
receiving by a console the FBG sensor-reflected optical signals from the optical interrogator, the console including one or more processors, memory, and
executable instructions stored in the memory that cause the console to perform various operations of the method upon execution of the instructions by the one-or-more processors;
converting the FBG sensor-reflected optical signals into converted electrical signals with optical signal-converter logic; and
determining in a real-time determination the one-or-more physical attributes associated with a heart, lungs, or both the heart and lungs from at least the converted electrical signals with physical attribute-determination logic.

14. The method of claim 13, further comprising extracting one or more extracted electrical signals from the converted electrical signals with signal-processing logic, the converted electrical signals including complex oscillations indicative of those experienced by the distal portion of the optical-fiber probe when disposed in a circulatory system of a patient and the one-or-more extracted electrical signals including one or more simple oscillations indicative of the one-or-more physical attributes associated with the heart, the lungs, or both the heart and lungs.

15. The method of claim 14, wherein the one-or-more physical attributes associated with the heart include heart rate, the physical attribute-determination logic configured to determine the heart rate directly from a simple oscillation from an extracted electrical signal of the one-or-more extracted electrical signals.

16. The method of claim 14, wherein the one-or-more physical attributes associated with the lungs include respiration rate, the physical attribute-determination logic configured to determine the respiration rate directly from a simple oscillation from an extracted electrical signal of the one-or-more extracted electrical signals.

17. The method of claim 16, further comprising correlating one or more measurements of the one-or-more physical attributes associated with the heart, the lungs, or both the heart and lungs by one or more measuring devices therefor with the one-or-more simple oscillations indicative of the one-or-more physical attributes associated with the heart, the lungs, or both the heart and lungs prior to determining the one-or-more physical attributes associated with the heart, the lungs, or both the heart and lungs in the real-time determination.

18. The method of claim 17, wherein the one-or-more physical attributes associated with the heart are selected from relative central blood pressure, right atrial pressure, right ventricular pressure, pulmonary artery pressure, and pulmonary artery wedge pressure.

19. The method of claim 17, wherein the one-or-more physical attributes associated with the heart include stroke volume.

20. The method of claim 17, wherein the one-or-more physical attributes associated with the lungs are selected from vital capacity, tidal volume, inspiratory capacity, expiratory reserve volume, inspiratory reserve volume, and total lung capacity.

21. The method of claim 17, further comprising:
establishing one or more physical attribute-baseline measurements through the one-or-more measurements of the one-or-more physical attributes; and
monitoring one or more physical-attribute baselines through the one-or-more simple oscillations to determine any deviations from the one-or-more physical-attribute baselines.

22. The method of claim 16, further comprising displaying the physical attributes associated with the heart, the lungs, or both the heart and lungs on a display screen, optionally, in one or more physical-attribute plots to facilitate historical analysis by an attending clinician.
